(19) United States
(12) Patent Application Publication
(10) Pub. No.: US 2002/0025527 A1
(43) Pub. Date: Feb. 28, 2002

(54) Methods and assays for diagnosing alzheimer's disease

(76) Inventors: Fiona Crawford, Tampa, FL (US); Michael Mullan, Tampa, FL (US)

Correspondence Address: Amy E. Rinaldo, Kohn & Associates, Suite 410, 30500 Northwestern Highway, Farmington Hills, MI, 48334, US

(21) Appl. No.: 09/898,206

(22) Filed: Jul. 3, 2001

Related U.S. Application Data
(60) Provisional application No. 60/215,907, filed on Jul. 3, 2000.

(51) Int. Cl. C12Q 1/68

(52) U.S. Cl. 435/6

(57) ABSTRACT

There is provided a method and assay for detecting if a subject, especially subjects over the age of 79, is at increased risk of developing late onset Alzheimer's disease (AD) by directly or indirectly detecting polymorphisms in the cystatin C gene (CST3) in the subject, and observing whether or not the subject is at increased risk of developing late onset AD by determining the alleles present at an AD associated CST3 polymorphism, wherein the presence of particular alleles at the CST3 polymorphism indicates said subject is at increased risk of developing late onset AD. Also provided is a method useful as an aid in determining the prognosis for late onset Alzheimer's Disease (AD) of a subject by detecting the presence or absence of an CST3 GG genotype in the subject, observing that (i) the subject's prognosis is more negative for late onset AD if the presence of the CST3 GG genotype is detected than if it is absent, or that (ii) the subject's prognosis is more positive for late onset AD if the CST3 GG genotype is absent than if it is detected. A method of detecting if a subject is at increased risk of developing late onset Alzheimer's disease by directly or indirectly determining the secreted levels of cystatin C in the subject and/or determining the intracellular levels of cystatin C in the subject.

### CROSS-RELATED REFERENCE SECTION

This application claims the benefit of priority under 35 U.S.C. Section 119(e) of United States Provisional Patent Application No. 60/215,907, filed Jul. 3, 2000, which is incorporated herein by reference.

### TECHNICAL FIELD

The present invention relates to methods for diagnosing Alzheimer's Disease (AD). More specifically, the present invention relates to methods of assessing an individual's risk for developing AD by screening individuals for the presence of variations/polymorphisms in the cystatin C (CST3) gene.

### BACKGROUND ART

According to the Alzheimer's Association, Alzheimer's disease accounts for more than 50 percent of the dementias seen in the general adult population. The estimated annual incidence of Alzheimer's disease is approximately 2.4 per 100,000 people age 40 to 60 and 127 per 100,000 people older than age 60. Estimates are that five percent of people over the age of 60 will have Alzheimer's neuropathology, and that this increases to 50 percent for the population age 85 and older. Current estimates by the National Institute of Aging are that more than four million Americans are affected by the disease and that this number will grow to 12 million over the next 20 years.

The disease usually begins after age 65, and risk of AD goes up with age. While younger people also may have AD, it is much less common. About 3 percent of men and women ages 65 to 74 have AD, and nearly half of those age 85 and older may have the disease. In other words, at age 60 and above you have a 1 in 10 chance of developing Alzheimer's and those aged 80 and above have a 1 in 4 chance of developing the disease. It is important to note, however, that AD is not a normal part of aging.

The new cause-of-death classification system also resulted in a significant shift in ranking for Alzheimer's disease. In 1998 Alzheimer's disease ranked 12^{th} among leading causes of death, but in 1999 it was ranked 8^{th}. The 44,507 deaths from Alzheimer's disease in 1999 surpassed the totals for other major causes of death, including motor vehicle accidents and breast cancer.

The apolipoprotein E gene (APOE) has been demonstrated to be a major risk factor for Alzheimer's Disease (AD) in Caucasian, Hispanic, African American and Asian populations^{1.2}, and odds ratios ranging from 2.0-7.7 have been reported for ε 4-carrying individuals¹. However, the magnitude of risk conferred by the ε4 allele is known to be age-dependent, appearing maximal in the 60-80 age group^{1,2,3,4,5}, and in at least one study, the OR for the ε4 allele in this age group is significantly higher than those in either the <60 or >79 age groups⁴. Such observations prompt the hypothesis that additional genetic risk factors other than APOE confer risk in the older and younger age groups. In the search for genes contributing risk for late-onset AD, candidate genes proposed either from clinical or neuropathological observations in AD patients or from the biological activities of the β-amyloid (Aβ) and apolipoprotein E (ApoE) proteins have been investigated.

The published literature on APOE from many AD case and control populations supports the fact that APOE e4-carrying genotypes contribute greatest risk for AD in the 60-80 age group, after which time the risk decreases considerably to the point at which APOE is no longer a risk factor for AD^{1,2}. Therefore, APOE-conferred risk cannot explain the observed increasing incidence of AD with age³¹.

AD and vascular dementia share common genetic risk factors, such as the ε4 allele of APOE^{6,7,8,9,10} and vascular factors such as hypertension, atrial fibrillation, diabetes and high plasma cholesterol level^{11,12,13,14,15}. These factors contribute to AD pathogenesis by modifying the risk of developing AD, altering clinical presentation, and changing the progression of the disease^{16,17,18}. The deposition of β-amyloid (Aβ) in the walls of cerebral vessels as Cerebral Amyloid Angiopathy (CAA) is one of the commonest abnormalities detected in AD brains at autopsy (83% of AD cases as assessed by CERAD¹⁹). Immunohistochemical studies in AD and CAA brains have demonstrated co-localization of Aβ with another amyloidogenic protein, cystatin C (also known as gamma-trace), within arteriolar walls^{20,21}.

Many positive associations between AD and putative genetic risk loci have been reported such as LRP³², A2M³³, VLDL³⁴, ACE³⁵, ACT³⁶, NOS³⁷, and BuCHe³⁸, but few have been replicated, and none as conclusively as APOE. However, APOE conferred risk cannot explain the observed increasing incidence of AD with age.

It would therefore be useful to determine a more accurate marker, method, and assay for determining the potential for acquiring late-onset Alzheimer's Disease.

### SUMMARY OF THE INVENTION

According to the present invention, there is provided a method and assay for detecting if a subject, especially subjects over the age of 79, is at increased risk of developing late onset Alzheimer's disease (AD) by directly or indirectly detecting polymorphisms in the cystatin C gene (CST3) in the subject, and observing whether or not the subject is at increased risk of developing late onset AD by determining the alleles present at an AD associated CST3 polymorphism, wherein the presence of particular alleles at the CST3 polymorphism indicates said subject is at increased risk of developing late onset AD. Also provided is a method useful as an aid in determining the prognosis for late onset Alzheimer's Disease (AD) of a subject by detecting the presence or absence of an CST3 GG genotype in the subject, observing that (i) the subject's prognosis is more negative for late onset AD if the presence of the CST3 GG genotype is detected than if it is absent, or that (ii) the subject's prognosis is more positive for late onset AD if the CST3 GG genotype is absent than if it is detected. A method of detecting if a subject is at increased risk of developing late onset Alzheimer's disease by directly or indirectly determining the secreted levels of cystatin C in the subject and/or determining the intracellular levels of cystatin C in the subject.

### BRIEF DESCRIPTION OF THE FIGURES

Other advantages of the present invention will be readily appreciated as the same becomes better understood by reference to the following detailed description when consider in connection with the accompanying drawings wherein:

FIG. 1 is a table showing the presence of CST3 and APOE1 genotype individuals who are eighty years of age or older and those who are less than eighty years of age;

FIGS. 2 A and B are tables showing the presence of CST3 and APOE1 alleles and genotype frequencies in individuals of Hispanic origin; and

FIG. 3 is a graph showing the genotype frequencies across age groups of the CST3 gene.

### DETAILED DESCRIPTION OF THE INVENTION

Generally, the present invention provides an assay for assessing an individual's risk of developing late-onset Alzheimer's Disease. The assay functions by screening samples, such as peripheral blood, for the presence of alleles present at the variations/polymorphisms of the CST3 gene. In addition, the invention provides supplement diagnostic strategies for treating Alzheimer's Disease by screening samples, such as such as peripheral blood, for the presence of alleles present at the polymorphisms of the CST3 gene.

Cystatin C is the most abundant extracellular inhibitor of cysteine proteases, and a mutation at position 68 (L68Q) of the protein gives rise to one of the familial cerebral amyloid angiopathies, hereditary cerebral hemorrhage with amyloidosis, Icelandic type (HCHWA-l)²². This autosomal-dominant form of amyloidosis is restricted to the small vasculature of the brain and is clinically characterized by recurrent strokes leading to an early death²³. Vascular deposition of cystatin C results in progressive loss of smooth muscle cells as cystatin C accumulates, leading to microvascular degeneration and cerebral hemorrhage²¹. However, no one has established a link between any of the above and the occurrence of Alzheimer's Disease.

In HCHWA-l patients the mutated protein is the main component of the amyloid deposits, and the pathogenetic mechanism is likely to be related to the in vitro observations of altered synthesis and secretion. A reduced rate of synthesis and secretion of cystatin C was demonstrated in cultured monocytes derived from patients carrying the mutation²⁴, while transfected mammalian cells have shown the mutant protein to form dimers of greater stability than wild type protein, resulting in reduced secretion and intracellular accumulation compared to wild type^{25,26}.

Applicants have investigated the occurrence of a coding polymorphism in the cystatin C gene (CST3) in 309 AD cases and 134 controls and find a significant interaction between CST3 genotype and age/age of onset on diagnosis of AD. More as specifically, in this case-control study a significant interaction between the CST3 GG genotype and age/age of onset, and a trend for the interaction between the APOE e4-carrying genotype and age/age of onset on risk for AD; but whereas the risk conferred by APOE decreases with age, the risk conferred by CST3 increases with age. In the 80+ age group, CST3 genotype contributes a greater than two-fold risk 2D for late onset disease while APOE genotype no longer confers significant risk for AD. The results show that the CST3 gene confers risk for AD at an age when APOE is no longer a major risk factor. The observed increase in the effect of CST3 GG genotype with age in the relatively young sample (age range 60-91; mean age of controls 76.1, mean age of onset of cases 75.1 yrs), further suggests that in older control and case populations, (e.g. those with a mean age/age of onset over 80 years), the contribution of CST3 GG genotype to the variance will be even more significant.

The CST3 genotype and polymorphisms can be detected via numerous methods know to those of skill in the art. For example, samples containing DNA, such as peripheral blood, are used to determine the CST3 genotype as follows. Peripheral blood samples containing DNA are tested for alleles present at polyrmorphisms in the CST3 gene. Peripheral blood samples are prepared using standard methods or the PUREGENE™ kit (gentra systems). Other tissue and/or cell samples can be taken, invasively or non-invasively. For example, mucosal can be taken from the mouth or nose by scraping, etc. Other methods known to those with skill in the art can also be used for detecting the presence of alleles present at the variations/polymorphisms of the gene. These methods include but are not limited to: determining genotype and allele frequencies of the polymorphism using allele specific polymerase chain reaction (PCR), restriction fragment length polymorphism (RFLP). Other methods that could be used would detect the amino acid change in the expressed protein, using antibodies, immunoprecipitation, Western blotting etc. Antibodies can be used to detect the amount of protein secreted or to determine the amount of protein secreted extracellularly versus the amount retained intracellularly.

Also provided by the present invention is the use of a marker for determining the risk of developing late-onset AD. The marker is preferably the genotype at the CST3 gene. The marker functions such that the presence of the marker in a tissue sample serves to indicate the risk of developing late-onset AD. The marker and method of using the same is useful as an aid in determining the prognosis for late onset Alzheimer's Disease (AD) of a subject by detecting the presence or absence of an CST3 GG genotype in the subject, observing that (i) the subject's prognosis is more negative for late onset AD if the presence of the CST3 GG genotype is detected than if it is absent, or that (ii) the subject's prognosis is more positive for late onset AD if the CST3 GG genotype is absent than if it is detected.

The invention is further described in detail by reference to the following experimental examples. These examples are provided for the purpose of illustration only, and are not intended to be liming unless otherwise specified. Thus, the invention should in no way be construed as being limited by the following examples, but rather should be construed to encompass any and all variations which become evident as a result of the teaching provided herein.

### EXAMPLES

### Example 1

### Materials and Methods

Study Group

Individuals comprising the sample population are selected and consist of 98 clinic and community based AD cases. Of this group, 40% are male with a mean age of onset at 76.8 years, the rang being 61-90 years. The control group of 129 persons contain 36% male subjects with a mean age of 75.7 years, range of 61-90 years.

Genotyping

Peripheral blood is genotyped at the CST3 locus. Peripheral blood is tested for the presence of CST3. Genotyping of the CST3+73 G/A polymorphism is carried out using primers: 5′ GCG GGT CCT CTC TAT CTA GC and 5′ ACT CAG GGC ATT CCC GGA CA is a polymerase chain reation with 1.5 mM Mg²⁺, 5% DMSO v/v, and 1 unit if DNA polymerase. PCR conditions: 94° C. for five minutes, followed by 35 cycles of 94° C. for thirty seconds, 55° C. for thirty seconds, and 72° C. for thirty seconds, with a final extension of time 72° C. for five minutes. This results in a 500 bp PCR product.

The preparation is digested with the restriction endonuclease SstII (or isoschisomes) at 37° C. for a minimum of four hours. This allows detection of the G/A polymorphism. In the presence of the A allele there is no digestion, while the G allele allows digestion of the PCR product to two fragements of 357 and 143 bp.

### Results

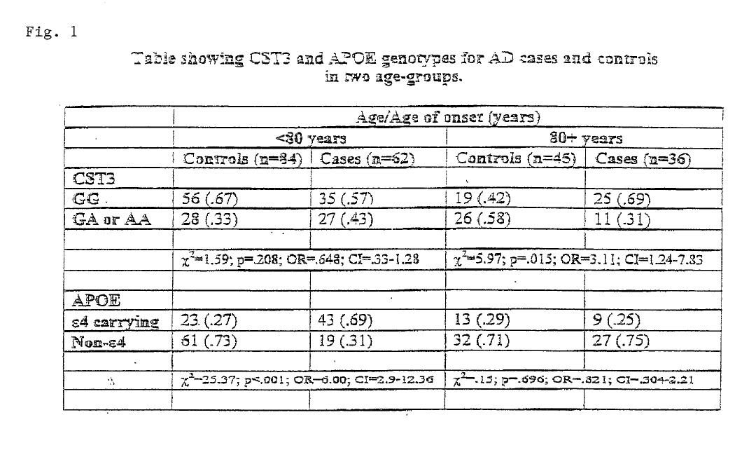

As shown by FIG. 1, stratification of the entire dataset on the basis of age/age of onset (30 years or 30+) followed by χ or χ² analysis demonstrated that in under 80 dataset CST3 genotype did not confer significant risk for diagnosis of AD (p=0.208) while APOE genotype conferred the expected high risk (p<0.001, OR=6). By contrast in the 80+ age group, APOE genotype no longer conferred increased risk for AD (p=0.696) while CST3 genotype was a significant risk factor (p=0.015) with an odds ration of 3.11.

### Statistical Analysis

Logistic regression analyses was performed and these revealed a significant 3-way interaction between APOE genotype (4 carriers versus non-4 carriers), CST3 genotype (GG versus G/A and AA genotype) and age/age of onset (p=0.034).

The sample was then extended by the genotyping of an additional 211 Caucasian clinic and community-based cases and 5 Caucasian controls. The mean age of onset of cases did not differ significantly from the mean age at normal memory screening for controls. The genotype distributions for both APOE and CST3 were in Hardy-Weinberg equilibrium for both case and control populations.

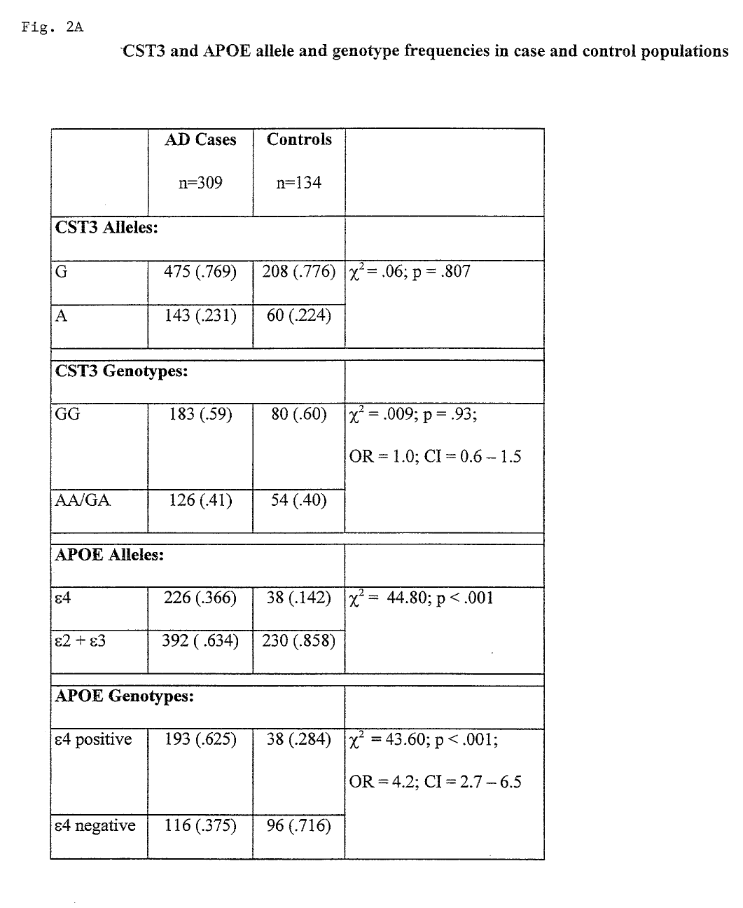

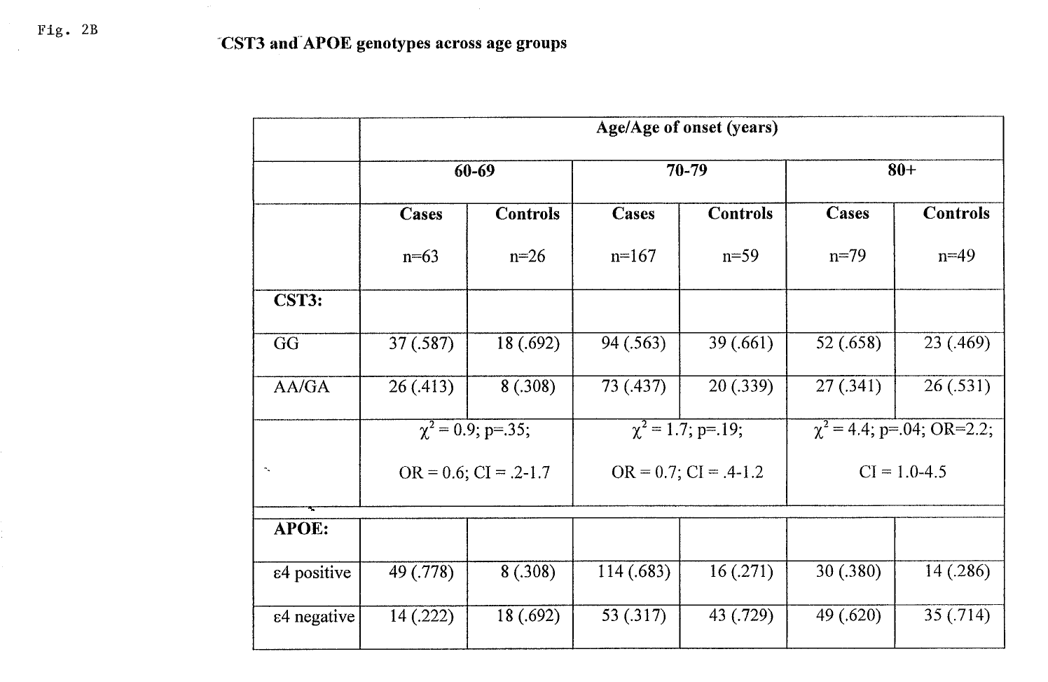

Analysis of APOE and CST3 allele and genotype distributions in the total dataset showed the expected association between APOE genotype and risk for AD, but no such association between CST3 and AD (FIG. 1). Logistic regression analyses again revealed a significant interaction between the CST3 genotype and age-of-controls/age-of-onset-of-cases (age/age of onset) on risk for AD. Stratification of the dataset based on age/age of onset revealed a CST3-genotype-dependent increased risk for AD in the 80+ group, (p=0.04) with an odds ratio of 2.18. In this age group APOE genotype did not confer increased risk for AD (p=0.28). In the lower two age groups (60-69 yrs and 70-79 yrs) APOE genotype was a risk factor while CST3 genotype was not (FIG. 2).

Together these data demonstrate that the genotype at the CST3 locus confers risk for AD, particularly in an older population. They further suggest that variation at the signal peptide polymorphism investigated in this study is the source of the conferred risk.

The effect of CST3 genotype on risk for AD was also investigated in an Hispanic population comprising 146 cases and 182 controls. As before, analysis of the total dataset demonstrated the expected risk associated with APOE genotype on AD diagnosis, but had no effect of CST3 genotype. Stratification of the Hispanic dataset (as per the Caucasian dataset) failed to demonstrate a significant effect of CST3 genotype on risk for AD, possibly as a consequence of the decreased numbers in the older dataset. However, a tendency is observed toward an increase of the GG genotype in cases versus controls in the older dataset (65% in the 80+group versus 59% in the 60-69 or 70-79 groups).

Together these data indicate that the genotype at the CST3 locus confers risk for AD in populations of different ethnicity, and indicates that pathogenic mechanisms involving the Cystatin C protein are consequent upon particular genotypes.

### Example 2

### Methods

The AD case sample consisted of 309 cases (56.3% women) recruited from both clinic (n=251) and community sources (n=58) and the control sample consisted of 134 subjects (64.2% women) recruited from the community. All AD cases in this study met NINCDS-ADRDA criteria for probable or possible AD. The AD clinic cases were subjects participating in a multi-center clinical drug trial and patients evaluated at the University of South Florida and the University of Miami Memory a Disorder Clinics (MDC). The community cases and controls were obtained from a community screen for dementia in the Dade county area of Florida. The community-based AD cases consisted of individuals who initially failed the screening evaluation (Mini Mental State Examination and evaluation of memory for recently acquired information and for overlearnt information (MMSE<27)), and were subsequently diagnosed following extensive examination at the Mount Sinai MDC in Miami. Individuals who were evaluated in the community-screening program and were found to be free of cognitive problems comprise the community controls (all had MMSE >27). Given the known relationship between cystatin C and cerebrovascular disease, individuals with known cerebrovascular abnormalities were excluded from our analyses.

All subjects were genotyped at the common polymorphism of APOE using primers and conditions as previously described²⁷. A protocol for detection of the CST3 polymorphism was previously described²⁸ and PCR was performed using these published primers, 1.5 mM Mg⁺⁺, 5% DMSO v/v and 1 unit of Biolase taq polymerase (Bioline) with the following PCR conditions: 94° C. for 5 minutes, followed by 35 cycles of 94° C. for 30 seconds, 55° C for 30 seconds, and 72° C. for 30 seconds, with a final extension time of 72° C. for 5 minutes. Digestion of the 500 bp PCR product with SstII to detect the G/A polymorphism resulted in an undigested 500 bp fragment in the case of the A allele while digestion to 357 bp and 143 bp fragments indicated the presence of the G allele.

Logistic regression was used to assess the main and interactive effects of A CST3 and APOE polymorphisms, gender, and age of controls or age of onset of cases (age/age of onset) on prediction of risk for AD. The χ² statistic was used to examine Hardy-Weinberg equilibrium and to further examine any interactions from the logistic regression analyses. Odds ratios corresponding to 95% confidence intervals (CIs) were calculated according to standard methods. Alpha levels were set at 0.05 for each analysis. Analyses were performed using SPSS for Windows release 9.0.1.

Using the χ² statistic preliminary analyses were conducted for case and control populations for the CST3 genotype distributions. The genotype distributions in case and control samples at the CST3 and APOE loci were found to be in Hardy-Weinberg equilibrium. As the AA genotype was infrequent in both case (5.5%) and control (4.5%) samples, analyses were conducted on the GG homozygous genotype versus the A-carrying genotypes. To provide sufficient power for interactions in logistic regression analyses, all analyses for APOE were conducted on ε4-carrying genotypes (ε4/ε4 and ε4/x) versus all non-ε4 carrying genotypes. There were no significant differences between APOE or CST3 genotype distributions between the community- and clinic-based cases (p>0.05) and so cases from these two sources were combined for these analyses. Comparison of age of onset of AD cases (75.1±6.2) and mean age of controls (76.1±7.4) revealed no significant difference (p=0.17).

### Results

For simple comparative purposes, results of χ² analyses of the CST3 and APOE genotype and allele frequencies are presented in FIG. 1. Hierarchical logistic regression analyses were used to examine the main and interactive effects of CST3 genotype, age/age of onset, and gender on risk for AD. These analyses revealed a significant interaction between CST3 genotype and age/age of onset (p=0.03), indicating increased risk for AD with increasing age. To examine this interaction in more detail, the sample was stratified into three age groups: 60-69, 70-79, and 80+ (FIG. 2). Analysis of the relationship of CST3 to AD in each of the three age groups by means of χ² analyses revealed increased risk for AD for GG genotypes in the age 80+ group (p=0.04, OR=2.18, Cl=1.05-4.51), but not for GG genotypes in the 60-69 and 70-79 age groups (p>0.10). No significant findings were obtained for other variables alone or in interaction.

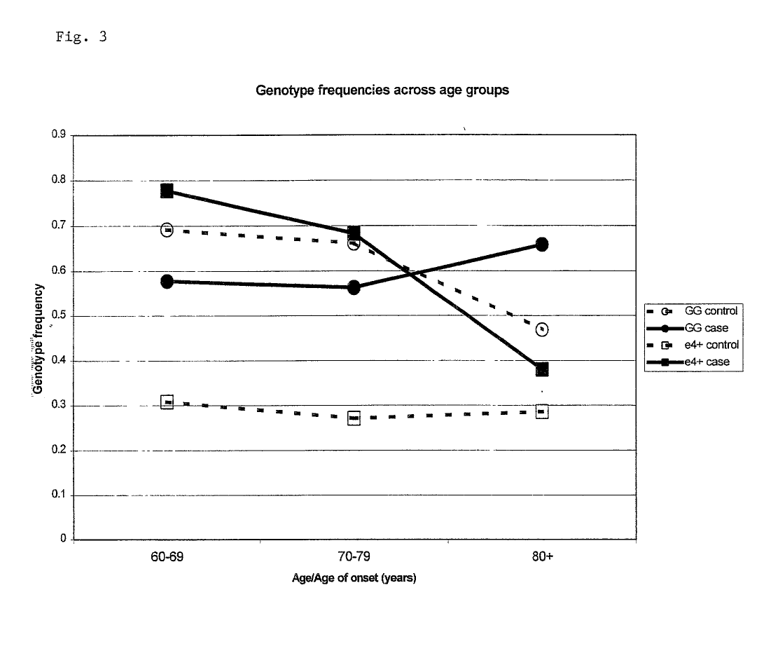

Hierarchical logistic regression analyses were used to examine the main and interactive effects of APOE genotype, CST3 genotype, and age/age of onset on risk for AD. As expected, the presence of the APOE ε4 allele was related to risk for AD (p<0.001), and there was no interaction between APOE and CST3 in producing risk (p=0.70). Although the interaction of the three variables was not significant (p=0.08), there was a tendency for the APOE ε4 risk to be greater for younger individuals (APOE genotype by age/age of onset interaction, p=0.06)—the reverse of the pattern found for the CST3 GG genotype. In FIG. 2 the analysis of the APOE-AD relationship is shown by χ² analyses in each of the three age groups previously described, which reveals increased risk for AD for the ε4-carrying genotypes in the 60-69 (p<0.001, OR=7.88, Cl=2.83-21.90) and 70-79 (p<0.001, OR=5.78, Cl=2.99-11.18) age groups, but not in the 80+ age group (p=0.28). In FIG. 3 there is plotted the frequency of the GG genotype in cases and controls, and the ε4+ genotype in cases and controls for each of these age groups to illustrate the transfer of risk for AD from APOE ε64+ to CST3 GG over the age of 80 years. As the ε2 allele has previously been suggested to be protective against AD, the analyses were repeated with the exclusion of the APOE ε2/4 samples (n=12). There were no substantive changes in the results, with the exception that the three-way interaction of APOE, CST3 and age/age of onset increased in effect size (p=0.01).

There are substantial differences in ORs for ε4-carrying genotypes between clinical and community-based samples, such that the recruiting bias introduced in clinic-based samples amplifies the APOE effects within that population. The analyses were therefore repeated for APOE and CST3 using only the community-based cases (n=58) versus the community-based controls (n=134). Despite the loss of power due to the reduced number of cases, there is observed a significant (p<0.05) three-way interaction between APOE, CST3 and age/age of onset. Stratification of this sample into the same three age groups described above revealed the same pattern of results for both APOE and CST3 as with the entire case sample.

### Discussion

In the AD case-control study there is evidence that a potentially functional polymorphism in the signal peptide-coding region of the CST3 gene is a risk factor for late onset AD. In the total dataset there is a significant interaction between the CST3 GG genotype and age/age of onset, and a trend for the interaction between the APOE ε4-carrying genotype and age/age of onset on risk for AD. Whereas the risk conferred by APOE decreases with age, the risk conferred by CST3 increases with age. In the 80+ age group, CST3 genotype contributes a greater than two-fold risk for late onset disease while APOE genotype no longer confers significant risk for AD. The published literature on APOE from many AD cases and control populations supports the fact that APOE ε4-carrying genotypes contribute greatest risk for AD in the 60-80 age group, after which time the risk decreases considerably to the point at which APOE is no longer a risk factor for AD^{1,2}. Therefore, APOE-conferred risk cannot explain the observed increasing incidence of AD with age⁻.

Although environmental factors associated with aging will play a more significant role in the disease process, familial studies suggest that additional genetic factors contribute risk for later onset disease⁵. The data suggest that the CST3 gene confers risk for AD at an age when APOE is no longer a major risk factor. The observed increase in the effect of CST3 GG genotype with age in the relatively young sample (age range 60-91; mean age of controls 76.1, mean age of onset of cases 75.1 yrs), further suggests that in older control and case populations, (e.g. those with a mean age/age of onset over 80 years), the contribution of CST3 GG genotype to the variance is even more significant.

Many positive associations between AD and putative genetic risk loci have been reported such as LRP³², A2M³³, VLDL³⁴, ACE³⁵, ACT³⁶, NOS³⁷, and BuCHe³⁸, but few have been replicated, and none as conclusively as APOE. One possible source of the discrepancies in results from different study populations is differences in age/age of onset. It may be that for some of the case-control association studies previously reported, positive associations have been found in older populations, which have failed to be replicated in follow-up studies in comparatively younger populations (compare, for example, BuChe studies^{39,40,41}). Typically the age/age of onset range in these late onset AD study populations ranges from 60-90 years, thus including individuals beyond the age of APOE-conferred risk, but such is the strength of the APOE effect that significant association is still detected in the total population. However, with genes of lesser effect (such as CST3) for which there is an age-specific association, analyses of the total study population without including age/age of onset as a variable will inevitably fail to detect the association (as is the situation with χ² analysis of CST3 genotype in the total dataset (Table 1)).

At this time, no functional effects of the cystatin C Ala→Thr polymorphism have been reported. Given its location, the penultimate amino acid of the signal peptide, it modifies the secretory processing pathway for cystatin C, by altering the trafficking of cystatin C to the endoplasmic reticulum or Golgi, or by disrupting cleavage of the signal peptide from the mature protein. Cystatin C is normally present intracellularly as monomers or inactive dimers which convert to the active monomeric form prior to secretion²⁶. In the case of production of mutant cystatin C in HCHWA-l, the mutant protein forms much more stable dimers than the wild type form, and although a small percentage of these dimers can be secreted the overall effect of the mutation is a reduction in cystatin C secretion and activity and an intracellular build up of CST3 aggregates^{25,26}. Similar, though less acute, effects result when the variation in the signal peptide reduces secretion. As with the relationship between Aβ and AD, it is difficult to predict whether or not aggregation of the protein or disruption of normal function is the primary pathologic mechanism and if loss or gain of function effects of this polymorphism on the protease inhibitor properties may have consequences in AD. It is also possible that the risk is actually conferred by a locus in linkage disequilibrium with the site of the Ala/Thr polymorphism; however, if it is another locus within CST3 then similar hypotheses regarding functionality would still apply.

Although cystatin C has previously been suggested to play a role in AD or cerebrovascular amyloidosis related to AD^{21,42,43}, a pathologic mode of action has not been reported and in general its involvement has appeared to be a consequence of ongoing disease mechanisms such as inflammatory or amyloidogenic consequences of soluble or aggregating Aβ. These findings suggest that cystatin C plays a more central role in the later-onset form of Alzheimer's disease when the consequences of more insidious mechanisms begin to take effect.

Throughout this application, various publications, including United States patents, are referenced by author and year and patents by number. Full citations for the publications are listed below. The disclosures of these publications and patents in their entireties are hereby incorporated by reference into this application in order to more fully describe the state of the art to which this invention pertains.

The invention has been described in an illustrative manner, and it is to be understood that the terminology which has been used is intended to be in the nature of words of description rather than of limitation.

Obviously, many modifications and variations of the present invention are possible in light of the above teachings. It is, therefore, to be understood that within the scope of the described invention, the invention may be practiced otherwise than as specifically described.

TABLE 1
Hispanic APOE and CST3 allele and genotype frequencies

| | AD Cases | Controls |
|---|---|---|
| | n = 146 | n = 182 |
| APOE Genotypes: | | |
| ε4 positive | 63 (.43) | 33 (.18) |
| ε4 negative | 83 (.57) | 149 (.82) |

χ² = 24.5; p < .001; OR = 3.43; Cl = 2.1 − 5.6

| CST3 Genotypes: | | |
|---|---|---|
| GG | 88 (.60) | 109 (.60) |
| AA/GA | 58 (.40) | 73 (.40) |

χ² = .005; p = .94; OR = 1.0; Cl = 0.65 − 1.6
Table 1: CST3 and APOE allele and genotype frequencies in case and control Hispanic populations. A comparison between cases and controls reveals no significant difference in CST3 genotype or allele distribution, as compared to the significant differences observed for APOE allele or genotype distribution.

TABLE 2
Hispanic CST3 genotype frequencies by age group

| | Age/Age of onset (years) | | | | | |
|---|---|---|---|---|---|---|
| | 60-69 | | 70-79 | | 80+ | |
| | Cases | Controls | Cases | Controls | Cases | Controls |
| | n = 56 | n = 93 | n = 64 | n = 76 | n = 26 | n = 13 |
| CST3: | | | | | | |
| GG | .59 | .61 | .59 | .59 | .65 | .54 |
| AA/GA | .41 | .39 | .41 | .41 | .35 | .46 |

| | | |
|---|---|---|
| χ² = .08; p = .78 | χ² = .00; p = .98 | χ² = .49; p = .49 |
| OR = 0.9 | OR = 1.0 | OR = 1.6 |
| Cl = .46 − 1.8 | Cl = .5 − 2.0 | Cl = .4 − 6.3 |

Table 2: CST3 genotypes across age groups in an Hispanic population. A comparison between cases and controls in each of the three age-groups reveals a trend towards an increase in the CST3 GG genotype in the oldest (80+) age group in cases versus controls. Numbers of samples in this age group are low, precluding the detection of significant differences in the analyses of cases versus controls.

### References

1. Farrer L A, Cupples L A, Haines J L, et al. Effects of age, sex, and ethnicity on the association between apolipoprotein E genotype and Alzheimer disease. A meta-analysis. APOE and Alzheimer Disease Meta Analysis Consortium. JAMA 1997; 278:1349-56.

2. Tang M X, Maestre G, Tsai W Y, et al. Relative risk of Alzheimer disease and age-at-onset distributions Caucasians, and Hispanics in New York City. Am J Hum Genet 1996; 58:574-84.

3. Frisoni G B, Manfredi M, Geroldi C, et al. The prevalence of apoE-epsilon4 in Alzheimer's disease is age dependent. J Neurol Neurosurg Psychiatry 1998; 65:103-6.

4. Bickeboller H, Campion D, Brice A, et al. Apolipoprotein E and Alzheimer disease: genotype-specific risks by age and sex. Am J Hum Genet 1997; 60:439-46.

5. Blacker D, Haines J L, Rodes L, et al. ApoE-4 and age at onset of Alzheimer's disease: the NIMH genetics initiative. Neurology 1997; 48:139-47.

6. Marin D B, Breuer B, Marin M L, et al. The relationship between apolipoprotein E, dementia, and vascular illness. Atherosclerosis 1998; 140:173-80.

7. Ji Y, Urakami K, Adachi Y, Maeda M, lsoe K, Nakashima K. Apolipoprotein E polymorphism in patients with Alzheimer's disease, vascular dementia and ischemic cerebrovascular disease. Dement Geriatr Cogn Disord 1998; 9:243-5.

8. Premkumar D R, Cohen D L, Hedera P, Friedland R P, Kalaria R N. Apolipoprotein E-epsilon4 alleles in cerebral amyloid angiopathy and cerebrovascular pathology associated with Alzheimer's disease. Am J Pathol 1996; 148:2083-95.

9. Treves T A, Bornstein N M, Chapman J, et al. APOE-epsilon 4 in patients with Alzheimer disease and vascular dementia. Alzheimer Dis Assoc Disord 1996; 10:189-91.

10. Isoe K, Urakami K, Sato K, Takahashi K. Apolipoprotein E in patients with dementia of the Alzheimer type and vascular dementia. Acta Neurol Scand 1996; 93:133-7.

11. Skoog I, Kalaria R N, Breteler M M. Vascular factors and Alzheimer disease. Alzheimer Dis Assoc Disord 1999; 13 Suppl 3:S106-14.

12.Schmidt R, Schmidt H, Fazekas F. Vascular risk factors in dementia. J Neurol 2000; 247:81-7.

13. Sparks D L. Coronary artery disease, hypertension, ApoE, and cholesterol: a link to Alzheimer's disease? Ann N Y Acad Sci 1997; 826:128-46.

14. Boston P F, Dennis M S, Jagger C. Factors associated with vascular dementia in an elderly community population. Int J Geriatr Psychiatry 1999; 14:761-6.

15. Kalmijn S, Launer L J, Oft A, Witteman J C, Hofman A, Breteler M M. Dietary fat intake and the risk of incident dementia in the Rotterdam Study. Ann Neurol 1997; 42:776-82.

16.Snowdon D A, Greiner L H, Mortimer J A, Riley K P, Greiner P A, Markesbery WR. Brain infarction and the clinical expression of Alzheimer disease. The Nun Study. JAMA 1997; 277:813-7.

17. Slooter A J, Tang M X, van Duijn C M, et al. Apolipoprotein E epsilon4 and the risk of dementia with stroke. A population-based investigation. JAMA 1997; 277:818-21.

18. Hofman A, Oft A, Breteler M M, et al. Atherosclerosis, apolipoprotein E, and prevalence of dementia and Alzheimer's disease in the Rofterdam Study. Lancet 1997; 349:151-4.

19. Ellis R J, Olichney J M, Thai L J, et al. Cerebral amyloid angiopathy in the brains of patients with Alzheimer's disease: the CERAD experience, Part XV. Neurology 1996; 46:1592-6.

20. Vinters H V, Nishimura G S, Secor D L, Pardridge W M. Immunoreactive A4 and gamma-trace peptide colocalization in amyloidotic arteriolar lesions in brains of patients with Alzheimer's disease. Am J Pathol 1990; 137:233-40.

21. Wang Z Z, Jensson O, Thorsteinsson L, Vinters H V. Microvascular degeneration in hereditary cystatin C amyloid angiopathy of the brain. APMIS 1997; 105:41-7.

22. Levy E, Lopez-Otin C, Ghiso J, Geltner D, Frangione B. Stroke in Icelandic patients with hereditary amyloid angiopathy is related to a mutation in the cystatin C gene, an inhibitor of cysteine proteases. J Exp Med 1989; 169:1771-8.

23. Jensson O, Gudmundsson G, Arnason A, et al. Hereditary cystatin C (gamma-trace) amyloid angiopathy of the CNS causing cerebral hemorrhage. Acta Neurol Scand 1987; 76:102-14.

24. Thorsteinsson L, Georgsson G, Asgeirsson B, et al. On the role of monocytes/macrophages in the pathogenesis of central nervous system lesions in hereditary cystatin C amyloid angiopathy. J Neurol Sci 1992; 108:121-8.

25. Bjarnadottir M, Wulif B S, Sameni M, et al. Intracellular accumulation of the amyloidogenic L68Q variant of human cystatin C in NIH/3T3 cells. Mol Pathol 1998; 51:317-26.

26. Benedikz E, Merz G S, Schwenk V, Johansen T E, Wisniewski H M, Rushbrook J I. Cellular processing of the amyloidogenic cystatin C variant of hereditary cerebral hemorrhage with amyloidosis, Icelandic type. Amyloid 1999; 6:172-82.

27. Wenham P R, Price W H, Blandell G. Apolipoprotein E genotyping by one-stage PCR. Lancet 1991; 337:1158-9.

28. Balbin M, Grubb A, Abrahamson M. An Ala/Thr variation in the coding region of the human cystatin C gene (CST3) detected as a SstII polymorphism. Hum Genet 1993; 92:206-7.

29. Crawford F, Freeman M, Town T, et al. No genetic association between polymorphisms in the Tau gene and Alzheimer's disease in clinic or population based samples. Neurosci Lett 1999; 266:193-6.

30. Crawford F, Abdullah L, Schinka J, et al. Gender specific association of the angiotensin converting enzyme (ACE) gene with Alzheimer's Disease. Neuroscience Letters 2000; 280:215-9.

31. von Strauss E, Viitanen M, De Ronchi D, Winblad B, Fratiglioni L. Aging and the occurrence of dementia: findings from a population-based cohort with a large sample of nonagenarians. Arch Neurol 1999; 56:587-92.

32. Lendon C L, Talbot C J, Craddock N J, et al. Genetic association studies between dementia of the Alzheimer's type and three receptors for apolipoprotein E in a Caucasian population. Neurosci Lett 1997; 222:187-90.

33. Blacker D, Wilcox M A, Laird N M, et al. Alpha-2 macroglobulin is genetically associated with Alzheimer disease. Nat Genet 1998; 19:357-60.

34. Okuizumi K, Onodera O, Namba Y, et al. Genetic association of the very low density lipoprotein (VLDL) receptor gene with sporadic Alzheimer's disease. Nat Genet 1995; 11:207-9.

35. Kehoe P G, Russ C, McIlory S, et al. Variation in DCP1, encoding ACE, is associated with susceptibility to Alzheimer disease. Nat Genet 1999; 21:71-2.

36. Kamboh M l, Sanghera D K, Ferrell R E, DeKosky S T. APOE*4-associated Alzheimer's disease risk is modified by alpha 1-antichymotrypsin polymorphism. Nat Genet 1995; 10:486-8.

37. Dahiyat M, Cumming A, Harrington C, et al. Association between Alzheimer's disease and the NOS3 gene. Ann Neurol 1999; 46:664-7.

38. Lehmann D J, Johnston C, Smith A D. Synergy between the genes for butyrylcholinesterase K variant and apolipoprotein E4 in late-onset confirmed Alzheimer's disease. Hum Mol Genet 1997; 6:1933-6.

39. Mcilroy S P, Crawford V L S, Dynan K B, et al. Butyrylcholinesterase K variant is genetically associated with late onset Alzheimer's disease in Northern Ireland. J Med Genet 2000; 37:182-5.

40. Kehoe P G, Williams H, Holmans P, et al. The butyrylcholinesterase K variant and susceptibility to Alzheimer's disease. J Med Genet 1998; 35:1034-5.

41. Crawford F, Fallin D, Suo Z, et al. The butyrylcholinesterase gene is neither independently nor synergistically associated with late-onset AD in clinic- and community-based populations. Neurosci Lett 1998; 249:115-8.

42. McCarron M O, Nicoll J A, Stewart J, et al. The apolipoprotein E epsilon2 allele and the pathological features in cerebral amyloid angiopathy-related hemorrhage. J Neuropathol Exp Neurol 1999; 58:711-8.

43. Haan J, Maat-Schieman M L, van Duinen S G, Jensson O, Thorsteinsson L, Roos R A. Co-localization of beta/A4 and cystatin C in cortical blood vessels in Dutch, but not in Icelandic hereditary cerebral hemorrhage with amyloidosis. Acta Neurol Scand 1994; 89:367-71., based on APOE genotypes among elderly African Americans.

What is claimed is:

1. A method of detecting if a subject is at increased risk of developing late onset Alzheimer's disease (AD) comprising directly or indirectly:
detecting polymorphisms in the cystatin C gene (CST3) in the subject; and
observing whether or not the subject is at increased risk of developing late onset AD by determining the alleles present at an AD associated CST3 polymorphism, wherein the presence of particular alleles at the CST3 polymorphism indicates said subject is at increased risk of developing late onset AD.

2. The method according to claim 1, wherein the detecting step is carried out by collecting a biological sample containing DNA from the subject, and then determining the sequence of DNA encoding the CST3 polymorphism in the biological sample.

3. The method according to claim 2, wherein the determining step is carried out by amplifying the DNA encoding the CST3 polymorphism.

4. The method according to claim 3, wherein the amplifying step is carried out by polymerase chain reaction.

5. The method according to claim 1, wherein the detecting step is carried out by collecting a CST3 sample from the subject, and then detecting the alleles present at the AD associated CST3 polymorphism in said CST3 sample.

6. The method according to claim 1, wherein the subject has previously been determined to have one or more factors indicating that such subject is afflicted with Alzheimer's disease.

7. A method useful as an aid in determining the prognosis for late onset Alzheimer's Disease (AD) of a subject, said method comprising:
detecting the presence or absence of an CST3 GG genotype in the subject; and
observing that (i) the subject's prognosis is more negative for late onset AD if the presence of the CST3 GG genotype is detected than if it is absent, or that (ii) the subject's prognosis is more positive for late onset AD if the CST3 GG genotype is absent than if it is detected.

8. The method according to claim 7, wherein said detecting step is carried out by collecting a biological sample containing DNA from the subject, and then detecting the presence or absence of DNA encoding CST3 GG genotype in the biological sample.

9. The method according to claim 7, wherein said detecting step is carried out by polymerase chain reaction or ligase chain reaction.

10. A method of detecting if a subject is at increased risk of developing late onset Alzheimer's disease comprising directly or indirectly:
determining an amount of cystatin C in the subject in which a change in the amount of intrecellular cystatin C indicates an increased risk of developing late onset Alzheimer's disease.

11. The method according to claim 10, wherein said determining step includes determining an amount of secreted and/or intracellular cystatin C.

12. The method according to claim 10, wherein said identifying step is carried out by collecting a cystatin C sample from the subject, and then detecting the amount of cystatin C in the sample.

13. The method according to claim 11, wherein said identifying step is carried out by isoelectric focusing.

14. The method according to claim 11, wherein said amplifying step is carried out by immunoassay.

15. The method according to claim 11, wherein said identifying step is carried out by immunoassay with an antibody that selectively binds cystatin C.

16. The method according to claim 10, wherein said subject has previously been determined to have one or more factors indicating that such subject is afflicted with Alzheimer's disease.

17. A method of detecting if a subject over the age of 79 years is at increased risk of developing late onset Alzheimer's disease (AD) comprising directly or indirectly:
detecting the alleles occurring at CST3 polymorphisms in the subject; and
observing whether or not the subject is at increased risk of developing late onset AD by observing if the presence of an AD associated alleles of the gene polymorphism are or are not detected, wherein the presence of particular alleles at the gene polymorphism indicate said subject is at increased risk of developing late onset AD.
